(19) United States
(12) Patent Application Publication
(10) Pub. No.: US 2009/0199904 A1
(43) Pub. Date: Aug. 13, 2009

(54) LOW SHEAR PUMPS FOR USE WITH BIOREACTORS

(76) Inventors: Guy R. Babbitt, Fort Collins, CO (US); Christopher W. Turner, Windsor, CO (US); Peter A. Letvin, Fort Collins, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/371,353

(22) Filed: Feb. 13, 2009

Related U.S. Application Data
(60) Provisional application No. 61/028,224, filed on Feb. 13, 2008.

(51) Int. Cl. G05D 7/06; C12M 3/00; C12M 3/04

(52) U.S. Cl. 137/2; 435/289.1; 435/292.1

(57) ABSTRACT

Embodiments of a pump system include a bioreactor containing a media with live algae cultures, a gas vent line, a pressurized gas line, a valve system in fluid communication with the gas vent line and the pressurized gas line, a fluid outlet, and a pump chamber in fluid communication with the bioreactor, the valve system, and the fluid outlet. According to such embodiments, the valve system is configured to switch fluid communication with the pump chamber between the gas vent line and the pressurized gas line, and a lowest level of the pump chamber is lower than a highest level of the bioreactor. Such embodiments may also include a first valve configured to permit one-way flow from the bioreactor to the pump chamber, and a second valve configured to permit one-way flow from the pump chamber through the fluid outlet.

### CROSS-REFERENCE TO RELATED APPLICATION

This application claims the benefit of U.S. Provisional Patent Application Ser. No. 61/028,224, filed on Feb. 13, 2008, which is incorporated herein by reference in its entirety for all purposes.

### TECHNICAL FIELD

Embodiments of the present invention relate generally to low shear pumps, and more specifically to low shear pumps for use with bioreactors.

### BACKGROUND

Media within a bioreactor often contains cultures of microorganisms. For example, media within a photobioreactor often contains algae cultures, and often needs to be pumped through, into, or out of the bioreactor for various reasons, including, for example, nutrient distribution, mixing, agitation, processing, and/or extraction. However, many species of algae cannot tolerate shear stresses that are normally encountered in conventional or existing pumping mechanisms. Traditional high volume liquid pumps, such as positive displacement pumps and centrifugal pumps, often produce unacceptably high shear forces that can stress, lyse and/or deflagelate algae and other microorganisms.

### SUMMARY

A pump system according to embodiments of the present invention includes a bioreactor with a media, a gas vent line, a pressurized gas line, a valve system in fluid communication with the gas vent line and the pressurized gas line, a fluid outlet, and a pump chamber in fluid communication with the bioreactor, the valve system, and the fluid outlet. According to such embodiments, the valve system is configured to switch fluid communication with the pump chamber between the gas vent line and the pressurized gas line, and a lowest level of the pump chamber is lower than a highest level of the bioreactor. Such embodiments may further includes a first valve configured to permit one-way flow from the bioreactor to the pump chamber and a second valve configured to permit one-way flow from the pump chamber through the fluid outlet.

According to some embodiments of the present invention, the first and/or second valves are check valves, such as, for example, thin film check valves, or solenoid valves. The bioreactor may be a photobioreactor, such as, for example, a thin film photobioreactor, and the media may contain algae, in some embodiments. The valve system may include a dual-position, three-way solenoid valve; alternatively, the valve system may include a first two-position, two-way solenoid valve in the gas vent line and a second two-position, two-way solenoid valve in the pressurized gas line, according to embodiments of the present invention.

According to some embodiments of the present invention, a highest level of the pump chamber is higher than a lowest level of the bioreactor, to permit liquid level equilibrium between the pump chamber and the bioreactor when the pump chamber is in fluid communication with the gas vent line. According to other embodiments of the present invention, the low shear pump may include two or more pump chambers controlled to permit substantially continuous flow from the bioreactor into one of the two pump chambers.

A method for pumping bioreactor media at low shear according to embodiments of the present invention includes connecting a pump chamber with a bioreactor containing media, placing a valve inline between the pump chamber and the bioreactor, the valve configured to permit one-way flow from the bioreactor to the pump chamber, arranging the bioreactor and the pump chamber such that a media level in the bioreactor is higher than a liquid level in the pump chamber, and venting the pump chamber to begin flow of the media from the bioreactor, through the valve, and into the pump chamber, such that the flow of the media is at least partially caused by a difference in head pressure between the bioreactor and the pump chamber. The vent of the pump chamber may also be closed, and a pressurized gas source applied to the pump chamber to begin flow of the media out of the pump chamber. Embodiments of such methods may further include connecting a second pump chamber with the bioreactor, placing a second valve inline between the second pump chamber and the bioreactor, the second valve configured to permit one-way flow from the bioreactor to the second pump chamber, arranging the bioreactor and the second pump chamber such that the media level in the bioreactor is higher than a second liquid level in the second pump chamber, and alternately venting the first pump chamber when the pressurized gas source is applied to the second pump chamber and venting the second pump chamber when the pressurized gas source is applied to the first pump chamber, to permit a substantially continuous flow of media out of the bioreactor.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a low shear pump as media enters a pump chamber from a bioreactor, according to embodiments of the present invention.

FIG. 2 illustrates the low shear pump of FIG. 1 in which the media level in the bioreactor has come to equilibrium with a liquid level in the pump chamber, according to embodiments of the present invention.

FIG. 3 illustrates the low shear pump of FIGS. 1 and 2 as compressed regulated gas enters the pump chamber, according to embodiments of the present invention.

FIG. 4 illustrates the low shear pump of FIGS. 1-3 in which the compressed regulated gas has pushed liquid from the pump chamber through the fluid outlet, according to embodiments of the present invention.

FIG. 5 illustrates a front elevation view of a single chamber low shear pump according to embodiments of the present invention.

FIG. 6 illustrates a front elevation view of a double chamber low shear pump, according to embodiments of the present invention.

FIG. 7 illustrates a plot of flow rate versus air pressure for the low shear air displacement pump of FIG. 5, according to embodiments of the present invention.

FIG. 8 illustrates a front and top perspective view of a single chamber low shear air displacement pump, according to embodiments of the present invention.

FIG. 9 illustrates a front and bottom perspective view of the single chamber low shear air displacement pump of FIG. 8.

FIG. 10 illustrates an exploded front perspective view of the single chamber low shear air displacement pump of FIGS. 8 and 9, according to embodiments of the present invention.

FIG. 11 illustrates a front perspective view of a dual chamber low shear air displacement pump, according to embodiments of the present invention.

FIG. 12 illustrates some components that may be used to construct the dual chamber low shear air displacement pump of FIG. 11, according to embodiments of the present invention.

FIG. 13 illustrates a step in the construction of the dual chamber pump of FIG. 11, according to embodiments of the present invention.

FIG. 14 illustrates a step in the construction of the dual chamber pump of FIG. 11, according to embodiments of the present invention.

FIG. 15 illustrates a step in the construction of the dual chamber pump of FIG. 11, according to embodiments of the present invention.

FIG. 16 illustrates a step in the construction of the dual chamber pump of FIG. 11, according to embodiments of the present invention.

FIG. 17 illustrates a step in the construction of the dual chamber pump of FIG. 11, according to embodiments of the present invention.

FIG. 18 illustrates a step in the construction of the dual chamber pump of FIG. 11, according to embodiments of the present invention.

FIG. 19 illustrates a pneumatic diagram of the dual chamber pump of FIG. 11, according to embodiments of the present invention.

FIG. 20 illustrates operation of the dual chamber pump of FIG. 11, according to embodiments of the present invention.

FIG. 21 illustrates a check valve during fluid insertion, according to embodiments of the present invention.

FIG. 22 illustrates a check valve during air insertion, according to embodiments of the present invention.

FIG. 23 illustrates a low shear pump similar to the low shear pump of FIGS. 8-10, according to embodiments of the present invention.

FIG. 24 illustrates a low shear pump in which the valve system includes two two-way solenoid valves, according to embodiments of the present invention.

FIG. 25 illustrates a low shear pump in which the valve system includes a three-port, two-position solenoid valve, according to embodiments of the present invention.

FIG. 26 illustrates a dual pump chamber low shear pump in which the valve system includes two two-way solenoid valves for each pump chamber, according to embodiments of the present invention.

FIG. 27 illustrates a dual pump chamber low shear pump in which the valve system includes a single three-port, two-position solenoid valve for each pump chamber, according to embodiments of the present invention.

FIG. 28 illustrates the dual pump chamber low shear pump of FIG. 26, including liquid level sensors within the pump chambers, according to embodiments of the present invention.

FIG. 29 illustrates a low shear pump in which the valve system is a three-port, two-position solenoid valve, and which includes two-position, two-way solenoid valves in the fluid entry line and the fluid outlet, according to embodiments of the present invention.

FIG. 30 illustrates a low shear pump with a pump chamber in series between two bioreactors, according to embodiments of the present invention.

FIG. 31 illustrates a front elevation view of a film photobioreactor and low shear pump according to embodiments of the present invention.

FIG. 32 illustrates an enlarged front elevation view of a pump chamber in fluid communication with adjacent photobioreactors via thin film check valves, according to embodiments of the present invention.

FIG. 33 illustrates a step for making a thin film check valve, according to embodiments of the present invention.

FIG. 34 illustrates an additional step for making a thin film check valve, according to embodiments of the present invention.

FIG. 35 illustrates an additional step for making a thin film check valve, according to embodiments of the present invention.

FIG. 36 illustrates an additional step for making a thin film check valve, according to embodiments of the present invention.

FIG. 37 illustrates an additional step for making a thin film check valve, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

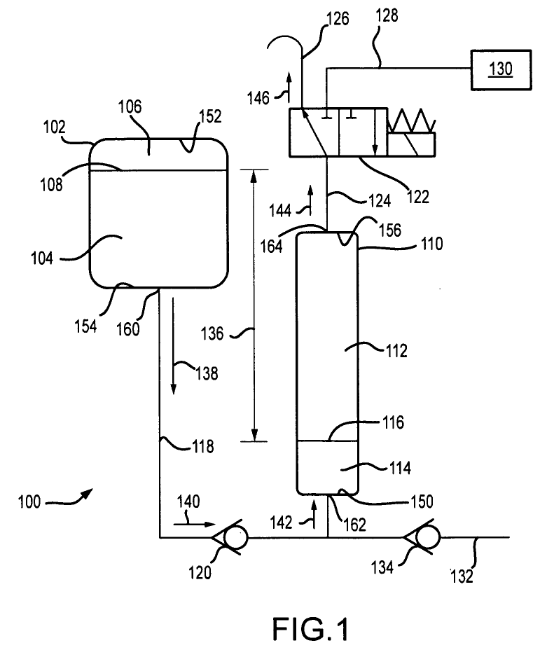

FIG. 1 illustrates a low shear pump 100 as media enters a pump chamber 110 from a bioreactor 102, according to embodiments of the present invention. The bioreactor 102 includes a liquid media 104 and a gas zone 106 above the media 104. The media 104 and gas zone 106 meet at the media liquid level 108. The pump chamber 110 includes a gas zone 112 and a liquid 114 which meet at liquid level 116. The pump chamber 110 and the bioreactor 102 may be vessels, chambers, or volumes of any size and/or shape.

The pump chamber 110 and the bioreactor 102 are in fluid communication via line 118. For example, the bioreactor 102 may include an opening 160 at which line 118 is connected to the bioreactor 102, and the pump chamber 110 may include an opening 162 at which the other end of line 118 is connected to the pump chamber 110. As used herein, the phrase “in fluid communication” is used in its broadest sense to refer to two or more elements between which fluid may flow, either directly or indirectly, either uni-directionally, bi-directionally, and/or multi-directionally, and either permanently or temporarily. For example, two reservoirs or chambers which are in fluid communication with each other may be connected by a hose, line, tube, conduit, valve, and/or shared opening.

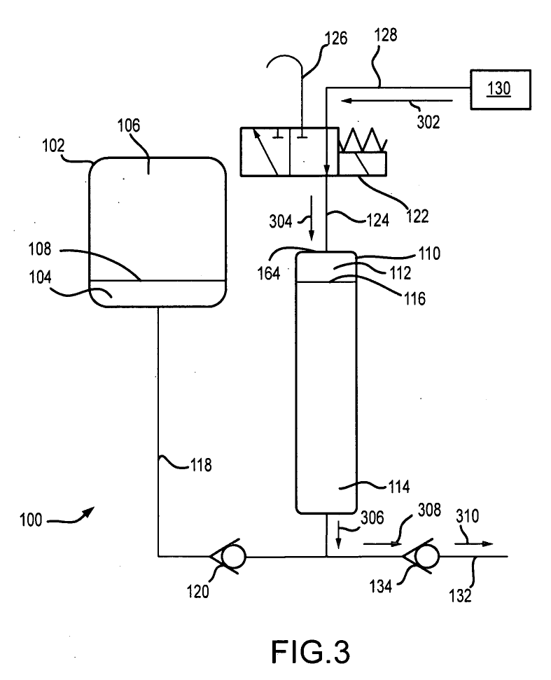

A valve 120 may be included inline with line 118, such that fluid is permitted to flow in only one direction through valve 120, from bioreactor 102 to pump chamber 110 in the direction indicated by arrow 140. Valve 120 may be, for example, a check valve or a solenoid valve. According to embodiments of the present invention, pump chamber 110 is in fluid communication with a valve system 122 via line 124. Line 124 may connect to the pump chamber 110 at an opening 164, according to embodiments of the present invention. The valve system 122 is in fluid communication with a gas vent line 126 and a pressurized gas line 128, which may in turn be in fluid communication with a pressurized and/or regulated gas source 130. According to some embodiments of the present invention, the valve system 122 is a three-port, dual-position solenoid valve which alternately places the pump chamber 110 in fluid communication with the gas vent line 126 in one position of the valve system 122 (the position depicted in FIG. 1) and the pump chamber 110 in fluid communication with the pressurized gas line 128 in the other position of the valve system 122 (the position depicted in FIG. 3).

Although FIG. 1 illustrates the valve system 122 as a three-port, dual position solenoid valve, based on the disclosure provided herein, one of ordinary skill in the art will recognize that various other types of valve systems may be used to achieve similar functionality. For example, two two-port, two-position solenoid valves may be used, one between the pump chamber 110 and the gas vent line 126 and the other between the pump chamber 110 and the pressurized gas line 128. Non-solenoid, or manual, valves may also be used, according to embodiments of the present invention. When solenoid valves are used, an optional controller (not shown) may be operatively associated with the solenoid valves (e.g. electrically coupled with the solenoid valves) to control the opening or closing of the valves, according to embodiments of the present invention.

The pump system 100 may further include a fluid outlet 132. Fluid outlet 132 may be a line 132 in fluid communication with the inlet line 118, or may alternatively be a stand-alone line 132 connected in fluid communication with the pump chamber 110 at a different location or opening. A valve 134 be included inline with line 132 such that fluid is permitted to flow in only one direction through valve 132, out of the pump chamber. Valve 134 may be, for example, a check valve or a solenoid valve. According to some embodiments of the present invention, the fluid outlet 132 is a simple opening in the pump chamber 110, and may be a shared opening with another chamber connected via valve 134.

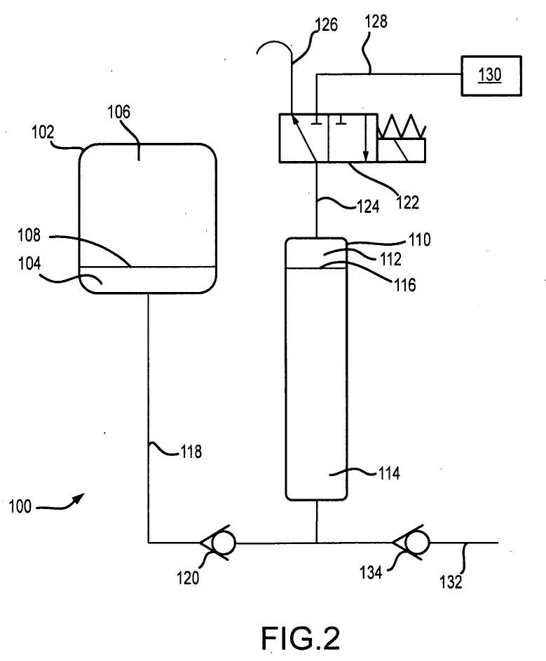

A difference in height 136 between the media 104 liquid level 108 and the pump chamber 110 liquid level 116 creates a head pressure between the bioreactor 102 and the pump chamber 110, according to embodiments of the present invention. This head pressure may be created by, for example, positioning the bioreactor 102 at a higher level (e.g. from the ground, when atmospheric pressure is used) than the pump chamber 110. Thus, to ensure that a positive head pressure can be developed between the bioreactor 102 and the pump chamber 110, the lowest level 150 of the pump chamber 110 should be lower than the highest level 152 of the bioreactor 102, according to embodiments of the present invention. An additional benefit may be achieved if the lowest level 154 of the bioreactor is lower than the highest level 156 of the pump chamber 110: such a configuration permits the media level 108 in the bioreactor 102 to reach equilibrium with the liquid level 116 in the pump chamber 110 (as illustrated in FIG. 2), which serves as a passive mechanism for halting fluid flow from the bioreactor 102 to the pump chamber 110 when the chamber 110 is vented (e.g. via gas vent line 126), according to embodiments of the present invention.

When the valve system 122 places the pump chamber 110 into fluid communication with the gas vent line 126, the bioreactor 102 and pump chamber are both exposed to atmospheric pressure, and the height difference 136 begins a flow of the media 104 through opening 160, through line 118, across the one-way valve 120, and into the pump chamber 110, in the direction of arrows 138, 140, 142, according to embodiments of the present invention. As the media 104 enters the pump chamber 110, the fluid 114 displaces the gas in the gas zone 112, which exits through opening 164, through line 124, through valve 122, and out of gas vent line 126 in the direction indicated by arrows 144,146, according to embodiments of the present invention. According to some embodiments of the present invention, instead of atmospheric pressure, the bioreactor 102 is a closed vessel having a higher pressure than a pressure at an outlet of the gas vent line 126. According to other embodiments of the present invention, the end of the gas vent line 126 is in fluid communication with the bioreactor 102, such as at or near the top level 152. In this way, the media 104, including any algae or other living microorganisms, are moved from the bioreactor 102 to the pump chamber 110 without a pump turbine blade or other mechanical device applying force or turbulence directly to the media 104, according to embodiments of the present invention.

The media 104 flowing from the bioreactor 102 to the pump chamber 110 is prevented from escaping through fluid outlet 132 by one-way valve 134, because the pressure at the downstream end of the line 132 is kept at a pressure equal to or greater than the pressure in the line 118, according to embodiments of the present invention. This may be achieved in some cases by connecting the downstream end of the line 132 in fluid communication with the bioreactor 102, such as at a location at or near the opening 160, according to embodiments of the present invention. According to other embodiments of the present invention, at least a portion of the media 104 flowing from the bioreactor 102 toward the pump chamber 110 flows across valve 134 and through fluid outlet 132 during the pump chamber filling cycle.

FIG. 2 illustrates the pump system 100 of FIG. 1 after the pump chamber 110 has been in fluid communication with the gas vent line 126 for a period of time (e.g. after the pump chamber filling cycle), during which the liquid level 116 in the pump chamber 110 has risen to the media level 108 in the bioreactor 102, according to embodiments of the present invention. At the moment in time illustrated in FIG. 2, fluid flow from the bioreactor 102 to the pump chamber 110 stops. According to some embodiments of the present invention, a liquid level sensor (not shown) is used in the pump chamber 110 to sense the liquid level 116 and close the pump chamber's 110 fluid communication with the vent line 126 and/or switch the pump chamber's 110 fluid communication to the pressurized gas line 128 at a predetermined liquid level. This may be done in order to stop the flow of media 104 through line 118 either before the media level 108 and liquid level 116 reach equilibrium, and/or to stop the flow of media 104 through line 118 for embodiments in which the lowest level 154 of the bioreactor 102 is higher than the highest level 156 of the pump chamber 110, to prevent over-filling of the pump chamber 110, for example. According to some embodiments of the present invention, the volume of media 104 in the bioreactor 102 is so large relative to the volume of the pump chamber 110 that a filling of the pump chamber 110 with media 104 from the bioreactor 102 causes little or almost no change in the media level 108 in the bioreactor 102.

As illustrated in FIG. 3, when the fluid communication between the pump chamber 110 and the gas vent line 126 is closed, and the fluid communication between the pump chamber 110 and pressurized gas line 128 is established, pressurized and/or regulated gas flows from the gas source 130, through pressurized gas line 128, through valve system 122, through line 124, through opening 164, and into the pump chamber 110 in the direction of arrows 302, 304, according to embodiments of the present invention. According to embodiments of the present invention, the gas source 130 is a compressed and/or pressurized gas supply that is at a pressure higher than the pressure in the pump chamber 110, and high enough to overcome any other pressure acting on the liquid in the pump. The gas source 130 is optionally regulated at the higher pressure to prevent fluctuations in pressure that might cause turbulence or other violent movement of the liquid 114 as it is pushed out of the pump chamber 110. As the pressurized gas enters the pump chamber 110, it displaces the liquid 114 and pushes it out through the fluid outlet 132, through valve 134, in the direction of arrows 306, 308, 310, according to embodiments of the present invention. The displaced liquid 114 is prevented from re-entering the bioreactor 102 through line 118 by the one-way valve 120.

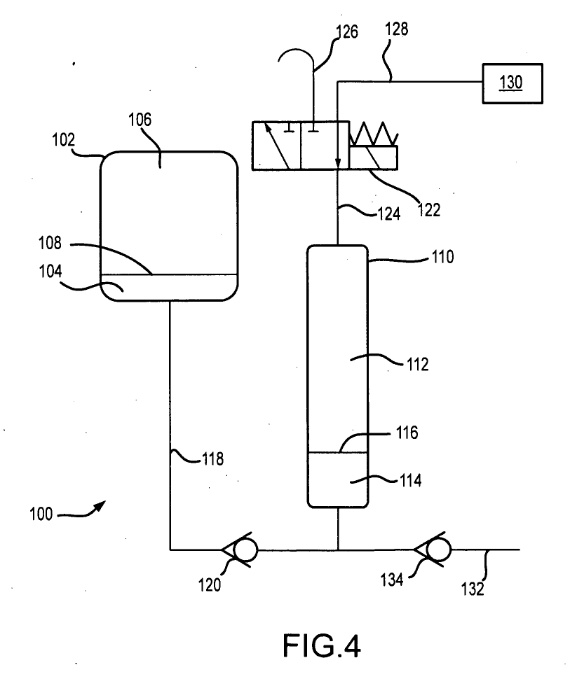

FIG. 4 illustrates the pump system 100 after the pressurized gas has displaced a substantial portion of the liquid 114 in the pump chamber 110, according to embodiments of the present invention. According to some embodiments of the present invention, a controller may be operatively associated with the valve system 122 to switch the pump chamber 110 from fluid communication with the pressurized gas line 128 back to fluid communication with the gas vent line 126 after a predetermined amount of time. According to yet other embodiments of the present invention, a controller may be operatively associated with one or more liquid level sensors (not shown) in the pump chamber 110 and with the valve system 122 to switch the pump chamber 110 from fluid communication with the pressurized gas line 128 back to fluid communication with the gas vent line 126 when the liquid level 116 has fallen to a predetermined liquid level. According to some embodiments of the present invention, the valve system 122 is controlled manually (e.g. by levers or knobs), by a timer, by a controller, and/or by a liquid level detection.

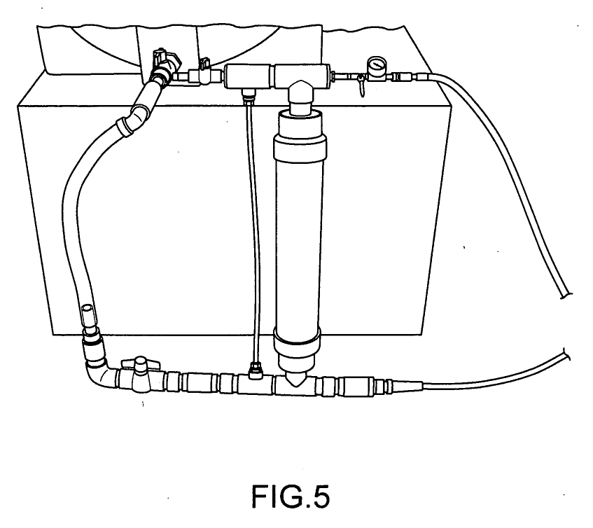

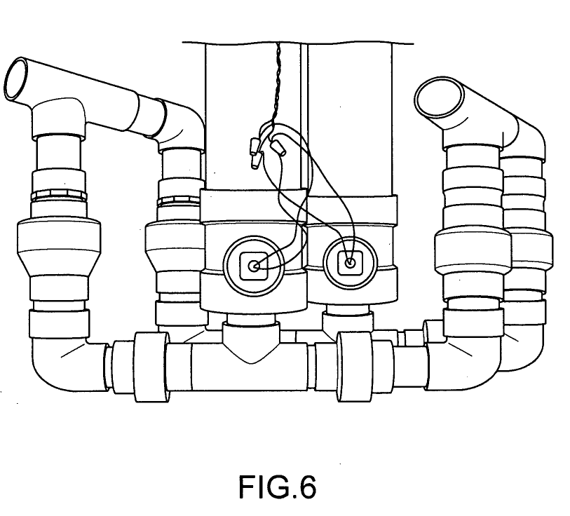

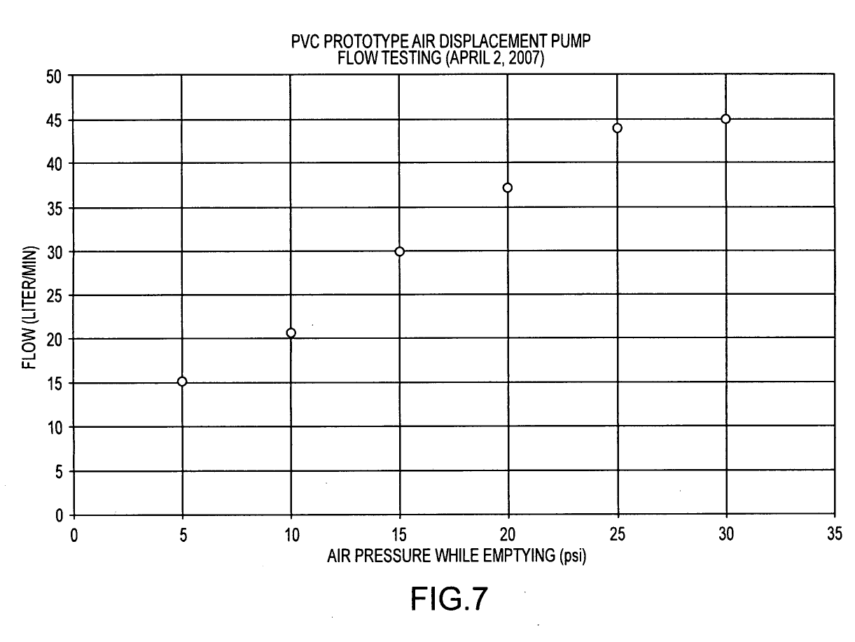

FIGS. 5 and 6 illustrate simple polyvinyl chloride (PVC) low shear air displacement pumps. FIG. 5 shows a single pump chamber design formed primarily from PVC tubing. The bioreactor/fluid reservoir is shown to the upper left of FIG. 5. FIG. 6 illustrates a double pump chamber design, also formed primarily from large diameter PVC tubing. In this design, the two pump chambers may operate consecutively, with one pump chamber filling while the other empties, allowing for an essentially continuous flow of liquid through the system. The pump operation is as described for FIGS. 1-4 above, with three-way valves at the top of the pump chamber controlling air movement and one-way check valves at the bottom of the pump chamber controlling liquid movement. The filling and emptying cycles of the PVC pumps are also as described with respect to FIGS. 1-4 above. FIG. 7 shows a plot of flow rate versus air pressure for the low shear air displacement pump illustrated in FIG. 5. Flow rate was approximately linear with air pressure, although flow rate began to level off at approximately forty-five to fifty liters per minute with this design. The two pump chamber design shown in FIG. 6 was tested at approximately 55-75 liters/min, but is capable of both higher and lower flow rates, according to embodiments of the present invention.

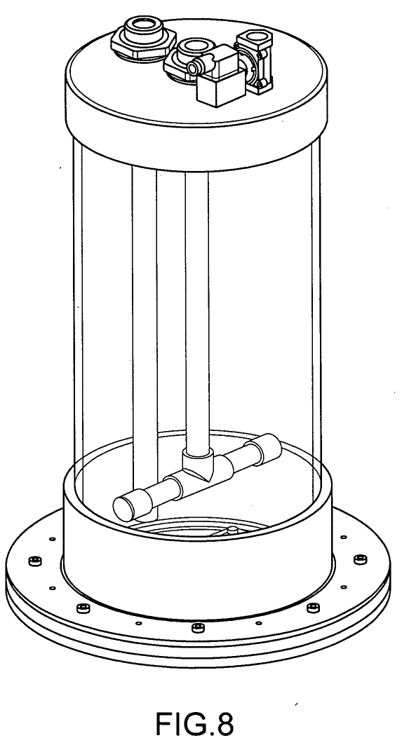

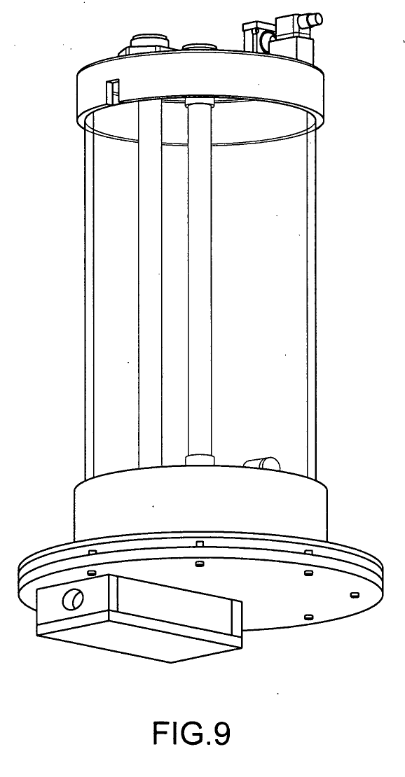

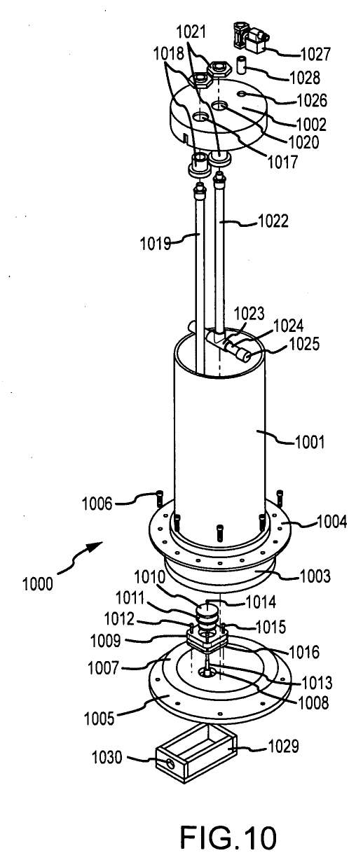

FIG. 8 illustrates a front and top perspective view of a single chamber low shear air displacement pump, according to embodiments of the present invention. FIG. 9 illustrates a front and bottom perspective view of the single chamber low shear air displacement pump of FIG. 8. FIG. 10 illustrates an exploded front perspective view of the single chamber low shear air displacement pump of FIGS. 8 and 9, according to embodiments of the present invention. The pump chamber 1001 was comprised of eight-inch diameter clear PVC pipe. Clear PVC was chosen to allow operation of the prototype to be monitored visually, but was not needed and other materials such as white or black PVC, other plastics, metals, or other materials could be used. The pump chamber was approximately twenty-four inches tall, a dimension that was selected such that the pump chamber was taller than the reservoir from which it was pumping. This was not a requirement, but was a feature that permitted equilibrium to be reached between the media 104 liquid level 108 and the pump chamber 110 liquid level 116, as described above, according to embodiments of the present invention. Other dimensions can be used with satisfactory performance. The pump chamber 1001 was capped and sealed on the top end by a PVC cap 1002 that had been cemented to the chamber using PVC primer and cement. On the other end of the chamber a PVC flange 1003 was also cemented to the pump chamber such that a clamp ring 1004 was used to secure the entire pump chamber assembly (PVC pipe, cap and flange). The clamp ring 1004 is bolted to a base plate 1005 using eight bolts 1006. The base plate used in this embodiment was made of ½ inch thick aluminum plate, but many other materials could be used.

While the pump chamber assembly 1000 of this embodiment was made with three separate pieces of PVC, many other means could be used to make the pump chamber assembly from as few as one piece such as, but not limited to, injection molding, rotomolding, casting, machining, etc. In the embodiment of FIG. 10, eight ¼ inch-20×1.25 LG UNC bolts 1006 were used to secure the clamp ring to the base plate which was tapped to accept the bolts. A gasket 1007 made of A35 durometer pure gum rubber, approximately ⅛ inch thick was trapped between the base plate and the flange and was compressed with the bolts to seal off the pump chamber. Other methods for sealing the pump chamber assembly to the base plate could be used such as, but not limited to, silicone sealants, other materials for the gasket, or eliminating the gasket altogether if the mating surface finishes and clamp loads are sufficient to prevent leakage without a gasket. In other embodiments the gasket could be eliminated and the pump chamber assembly 1000 could be permanently attached to the base plate with means such as, but not limited to, welding, adhesives, other fasteners, or making the base plate part of the pump chamber assembly 1000.

The base plate had a hole machined in 1008 it such that when attached to the pump chamber assembly there was a passage way to allow fluid to pass from below the base plate into the pump chamber assembly. In this embodiment the hole was approximately 1.75 inches in diameter. A check valve assembly was attached to the base plate to allow fluid to flow from below the base plate up into the pump chamber assembly, but not from the pump chamber assembly to below the base plate via hole 1008. In this embodiment the check valve assembly consisted of a check housing 1009, a check disc 1010, a check disc seal 1011, a second disc to support the seal 1012, a shaft that the check disc attached to 1013, a nut 1014 to compress and trap the check disc, the check disc seal, and the second disc to the shaft. The shaft can move axially to allow the check seal to contact the check housing and seal off fluid (e.g. in a closed position), or to an open position where there was a gap between the seal and the housing, allowing fluid to pass through from below the base plate into the pump chamber.

In the embodiment of FIG. 10, the check valve assembly was attached to the base plate using four stainless steel ¼ inch-20×1 inch LG UNC bolts 1015. The base plate was tapped to accept the bolts. A gasket 1016 was used to seal the check valve plate and the base plate. In this embodiment the gasket was made of seventy-durometer VITON®, but other embodiments could use other gasket materials or leave the gasket out altogether. Pump designs with and without gaskets have been successfully tested. Other embodiments could use different materials for the mounting bolts, or use entirely other means to attach the check valve to the base plates such as, but not limited to, adhesives, snap fit, clip retainers, rivets, snap connectors, and/or welding.

The check assembly was mounted such that gravity would move the check assembly to the closed position, and fluid forces would be used to lift the check valve to the open position. Often check valves use a spring to move the check valve to the closed position, and such a spring could be used with the embodiment illustrated in FIGS. 8-10. According to some embodiments of the present invention, such a spring may not be necessary due to the mounting of the valve at the bottom of the pump chamber. Both designs with and without springs have been tested.

The top cap 1002 of the pump chamber 1001 had three holes drilled into it. The first hole 1017 was approximately 1.4 inches in diameter and had a bulkhead connector 1018 secured in it. An exhaust tube 1019, in this case made of ½ inch schedule 40 PVC tubing, was connected to the bulkhead and was approximately twenty-two inches long such that it extended down inside the twenty-four inch tall pump chamber near to, but not touching, the base plate. Alternate embodiments use different materials or dimensions for the exhaust tube, including designs in which the end of the tube not connected to the bulkhead is connected to the check valve assembly and holds the check assembly in place, eliminating the need for other means of mounting the check assembly. The exhaust tube served to allow liquid to leave the pump chamber, according to embodiments of the present invention.

The second hole 1020 in the top cap 1002 was also approximately 1.4 inches in diameter and had another bulkhead connector 1021 mounted in it. This hole was used to connect the pump chamber to a source of higher pressure gas. In this embodiment the gas used was air from a two stage axial blower set to operate at approximately sixty inches of water pressure. The bulkhead connector had an air inlet tube 1022 attached to it, extending into the pump chamber such that the air was introduced at or near the bottom of the pump chamber to cause the gas to bubble, or sparge, through the liquid. In this embodiment, the bottom of the air inlet tube was connected to a ½ inch PVC “Tee” connector 1023, which was attached to two short, approximately 3 inch long, pieces of PVC tube 1024, both with ½ inch PVC caps 1025 attached to the other end. Each short PVC tube had approximately five ⅛ inch diameter holes drilled crosswise through the wall to allow the gas to escape from inside the tube to inside the pump chamber. In this embodiment the PVC pieces were cemented together using PVC primer and cement.

The pump chamber cap 1002 had a third hole 1026 in it that was tapped to ½ inch NPT. A two-position, two-way solenoid valve assembly 1027 was connected to the hole with a ½ inch NPT pipe nipple 1028 and teflon tape. The control valve was used to control the flow of higher pressure gas inside the pump chamber to the outside atmosphere or another collection reservoir. Alternate embodiments were built and tested that used other valve configurations, including a two-position three-way valve that not only controlled the flow of gas out of the chamber, but also the flow into the chamber. Another embodiment tested used two two-position, two-way valves that allowed the flow of gas into and out of the pump chamber.

The baseplate in this embodiment was connected to an inlet manifold 1029 made in this case of ½ inch aluminum plates welded together to form a box, which was in turn welded to the base plate. The inlet manifold in the preferred embodiment had a hole 1030 drilled in it such that a connector could be attached for fluid movement. Based on the disclosure provided herein, one of ordinary skill in the art will recognize that many different designs, configurations and materials could be used for the inlet manifold 1029.

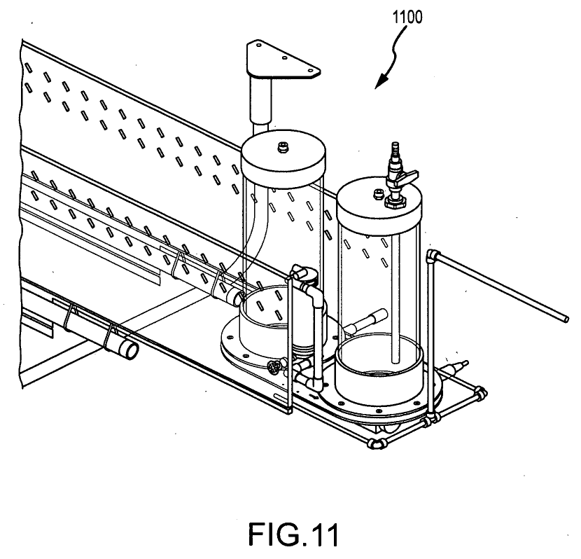

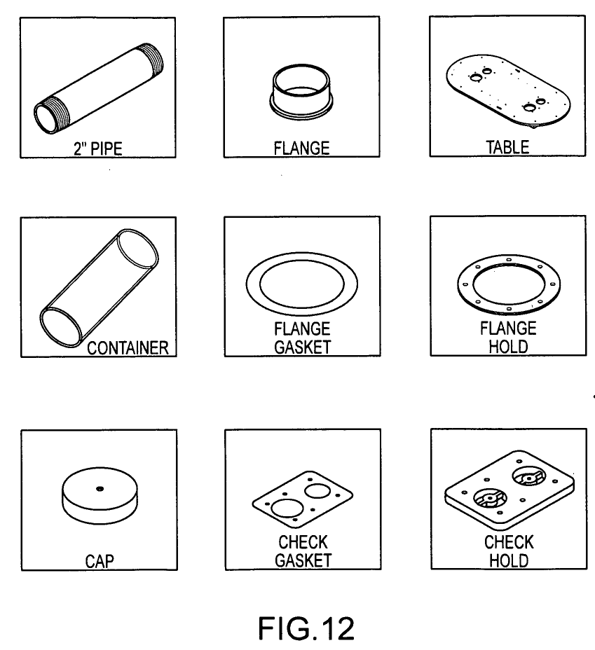

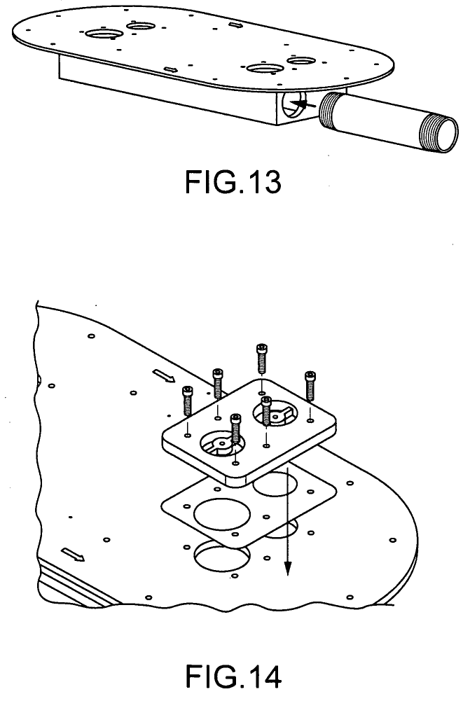

Another embodiment of a low shear air displacement pump 1100 is shown in FIG. 11, which shows a dual pump chamber design. In this embodiment, the pump chambers may be constructed of PVC. FIG. 12 illustrates various components that may be used to construct the dual pump chamber low shear air displacement pump 1100, according to embodiments of the present invention. The table (or base plate) may be composed of aluminum in order to minimize corrosion; alternatively, the base plate may be made of polycarbonate or other materials. FIGS. 13-18 illustrate various steps in making air displacement pump 1100, according to embodiments of the present invention. As illustrated in FIG. 13, the threads of a two-inch pipe may be covered with Teflon® tape and screwed tightly into the openings of the table. As illustrated in FIG. 14, the gasket may be lined up with holes in the table top; the check valves may be assembled into the check hold before fastening; the check hold may be placed onto the gasket, making sure the downward-facing check valve is inserted into the larger of the holes, according to embodiments of the present invention. The check hold may be tightened by hex cap screws.

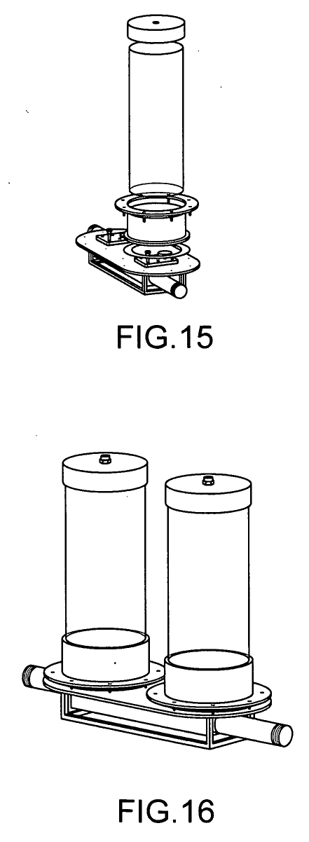

As illustrated in FIG. 15, PVC primer and cement may be applied to the area indicated, and the container may be fastened to the flange and the cap may be fastened to the container. Large pressure may be applied to seal the parts. The gasket may be centered and the flange placed to make maximum contact, and the flange hold may be slid over before the screws are tightened into openings in the table. As shown in FIG. 16, Teflon® tape may be applied to hose inserts on the top of the cap and screwed in tightly, such that the completed pump structure resembles that of FIG. 16.

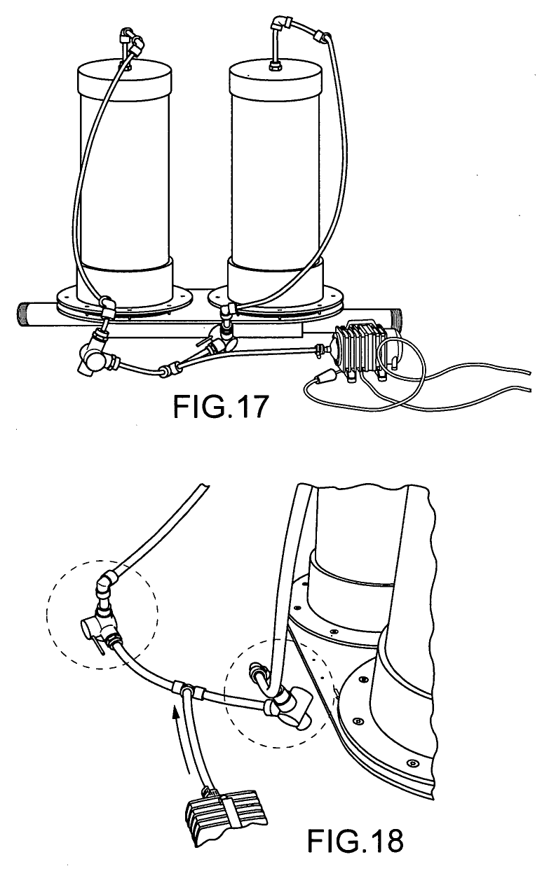

The air lines, which may be made out of ½ inch outer diameter plastic tubing, may be plugged directly into the hose connects on the top of the containers, as shown in FIG. 17. If the pump is mounted low, the pipe direction may be turned 180 degrees, according to embodiments of the present invention. The line from a compressed gas source may be connected to a T-connector to receive air in two directions, and lines from each of ends of the T-connector may then be connected to the three-way valves, which are circled in FIG. 18. One end of each three-way valve is connected to the pump or compressed air source and one end is open to atmosphere, and one valve port is in fluid communication with the pump chamber. Connectors may be used to plumb air to the hose insert, according to embodiments of the present invention.

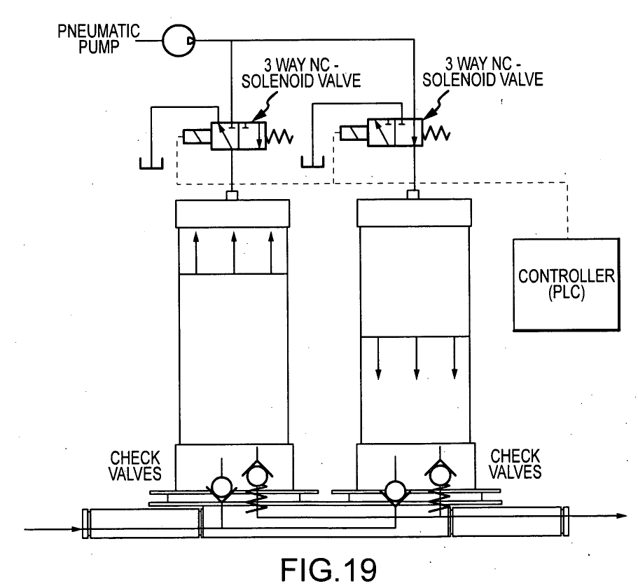

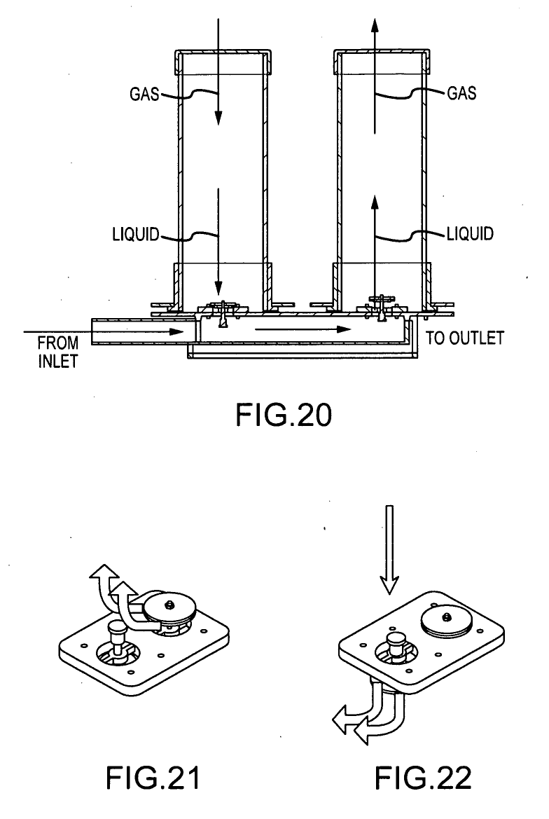

FIG. 19 illustrates a pneumatic diagram of the dual chamber pump 1100 of FIG. 11, according to embodiments of the present invention. FIGS. 20-22 illustrate how the pump 1100 works, according to embodiments of the present invention. According to some embodiments of the present invention, the pump is placed on sand so that the inlet pipe is lower than the top of the water line in the bioreactor (e.g. a photobioreactor bag). Using liquid weight and liquid head pressure, the liquid will tend to level out and force the inlet check valve open. Liquid contained in the outlet chamber will keep the outlet check valve closed. Closing the three-way valve feeds pressurized air into the pump chamber and displaces the fluid (e.g. water) downwardly, thereby closing the inlet check valve and pushing the outlet check valve open and emptying the pump chamber of liquid. Once the pump chamber is empty, the three-way valve may be opened again to permit the pressurized air to escape and fluid to fill through the inlet check valve from the bioreactor, according to embodiments of the present invention. FIG. 21 illustrates the pump open to vent air, during which the intake check valve permits liquid to pass and the pressure height in combination with a spring keeps the outlet check valve closed, according to embodiments of the present invention. FIG. 22 illustrates the pressurization of the pump chamber with compressed air to force the fluid downward, during which the weight of the liquid closes the inlet check valve and opens the outlet check valve, and liquid empties the pump chamber, according to embodiments of the present invention.

According to embodiments of the present invention, the low shear air displacement pump 1100 runs at fifty-two second cycles, with a head difference of seventeen inches, such that with an eight inch diameter pump chamber, the pump 1100 achieves an instantaneous flow rate of 4.27 gallons per minute.

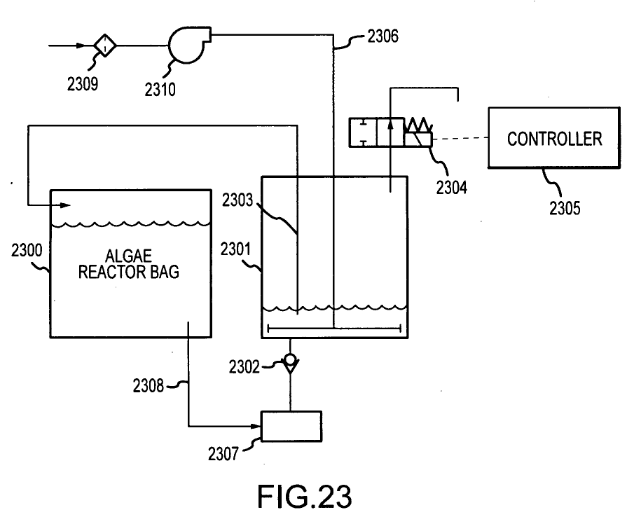

FIG. 23 shows an embodiment of a low shear air displacement pump, comprised of a pump chamber 2301, a check valve assembly 2302, an exhaust tube 2303, a control valve 2304, a controller or timing circuit 2305 to control the operation of the control valve, a gas inlet tube 2306 an inlet manifold 2307 and a line or connector 2308 that connects a photobioreactor 2300 (for example, an algal growth chamber as described in U.S. patent application Ser. No. 11/871,728, filed Oct. 12, 2007, which is incorporated herein by reference in its entirety) to the inlet manifold. Other embodiments can use two or more check valves, multiple control valves, level switches or other means to determine the pump chamber level, and may use more than one pump chamber to form a multi cylinder version.

In operation, the pump assembly is located adjacent to the photobioreactor so that the bottom of the pump inlet chamber 2307 in some cases may be level with or below the bottom of the photobioreactor growth chamber (labeled “Algae Reactor Bag” in FIG. 23). In the embodiment shown, the pump chamber 2301 is sized so that the top of it is higher than that of the maximum liquid level in the photobioreactor growth chamber. This prevents the pump chamber from overfilling, thus making operation simpler and more robust. With this orientation, control valve 2304 can be opened to allow any gas coming in through the gas inlet line 2306 to escape (e.g., by venting to atmosphere), causing fluid from the photobioreactor growth chamber to flow through the inlet line or connector 2308 into the inlet manifold 2307 and through check valve 2302 into the pump chamber 2301. If given sufficient time, the level in the pump chamber 2301 will be equal to that of the photobioreactor growth chamber. Once the fluid levels have stabilized and there is no flow through check valve 2302, gravity will cause the check valve to move to the closed position. In such a configuration, the flow areas through the gas inlet line circuit 2306 and the control valve circuit 2304 are sized such that the flow leaving the pump chamber 2301 greatly exceeds that of the gas flow coming in through the gas inlet line 2306, according to embodiments of the present invention.

According to some embodiments of the present invention, air is used for the gas and passes through a filter 2309 and is continuously delivered to the pump chamber through the gas inlet line 2306 and bubbled through the fluid. Such continuous sparging of the fluid may assist in removing dissolved gases such as oxygen from the fluid and keeping any algae or other suspended matter in suspension. This design also makes the control of the device simpler and more robust, as there are less valves to control.

After a period of time determined by the control unit 2305, the control valve 2304 is closed, preventing gas entering the pump chamber through the gas inlet lines 2306 from exiting the pump chamber through control valve 2304. As gas enters the pump chamber 2301 through gas inlet line 2306 without any means to leave the pump chamber the pressure in the pump chamber will start to increase. This increase in pressure will start to push fluid in the pump chamber 2301 through the exhaust tube 2303 and to the pump outlet. If control valve 2304 is left on long enough and the pressure in the gas source is sufficiently high the pump chamber can be evacuated to approximately the level of that of the lower end of the exhaust tube 2303. By controlling the timing of the control valve, and the pressures in the gas inlet line the pump chamber can be evacuated to varying levels in varying amount of time thus making the design a variable displacement and variable flow rate device.

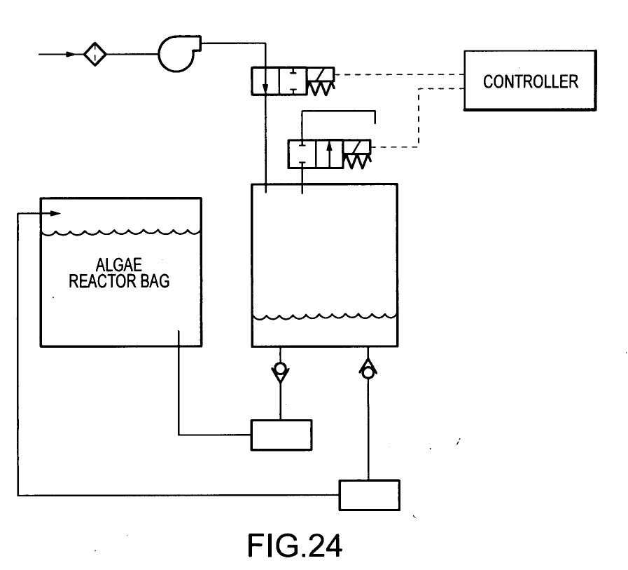

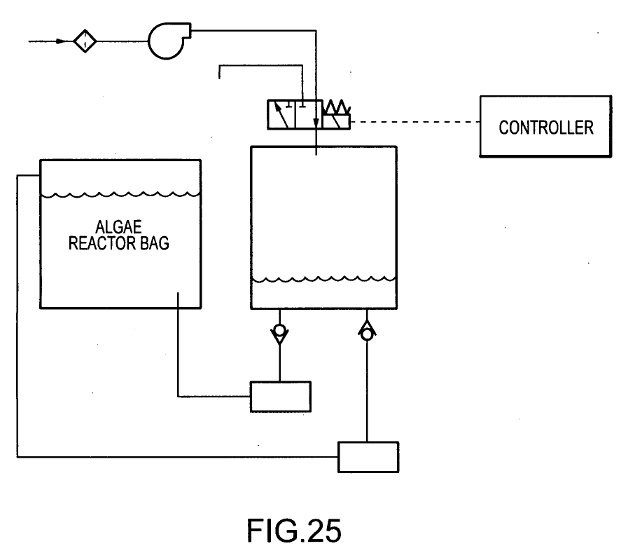

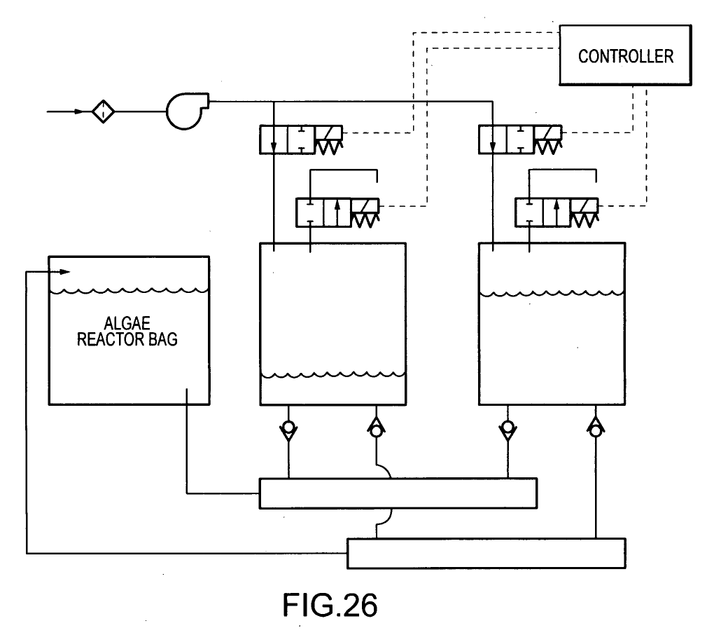

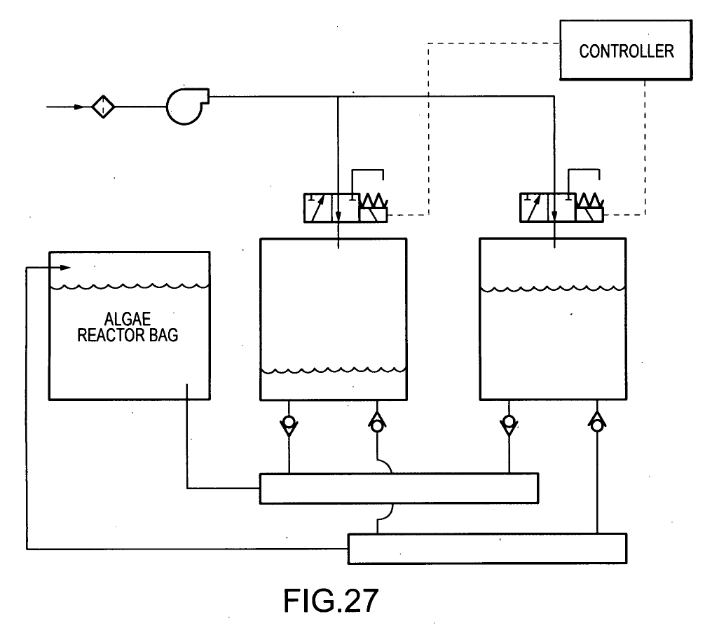

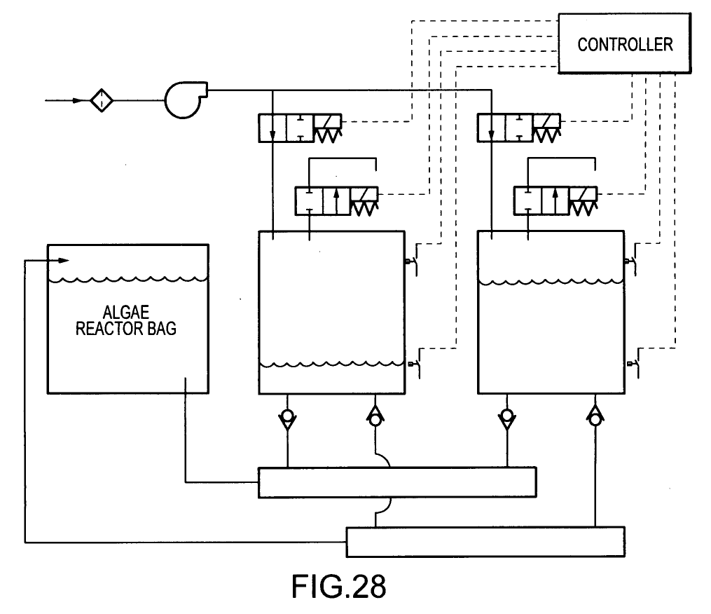

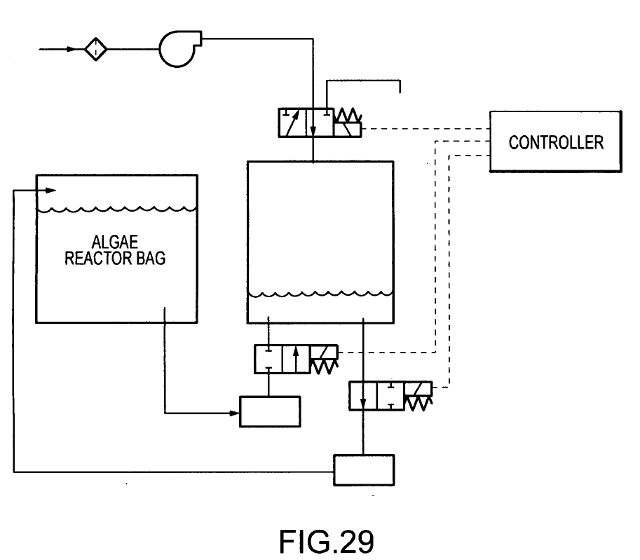

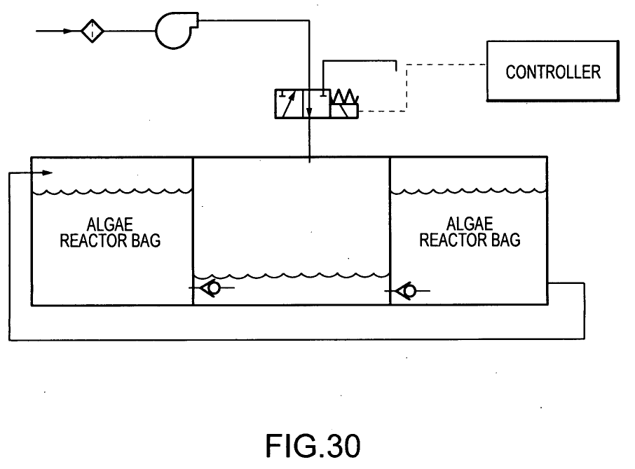

FIGS. 24-30 illustrate alternative embodiments of low shear air displacement pumps. Such embodiments operate generally according to principles described above with respect to the embodiments illustrated in FIGS. 1-4, 11, and 23. FIG. 24 illustrates a low shear pump in which the valve system includes two two-way solenoid valves, according to embodiments of the present invention. FIG. 25 illustrates a low shear pump in which the valve system includes a three-port, two-position solenoid valve, according to embodiments of the present invention. FIG. 26 illustrates a dual pump chamber low shear pump in which the valve system includes two two-way solenoid valves for each pump chamber, according to embodiments of the present invention. FIG. 27 illustrates a dual pump chamber low shear pump in which the valve system includes a single three-port, two-position solenoid valve for each pump chamber, according to embodiments of the present invention. FIG. 28 illustrates the dual pump chamber low shear pump of FIG. 26, including liquid level sensors within the pump chambers, according to embodiments of the present invention. FIG. 29 illustrates a low shear pump in which the valve system is a three-port, two-position solenoid valve, and which includes two-position, two-way solenoid valves in the fluid entry line and the fluid outlet, according to embodiments of the present invention. FIG. 30 illustrates a low shear pump with a pump chamber in series between two bioreactors, according to embodiments of the present invention.

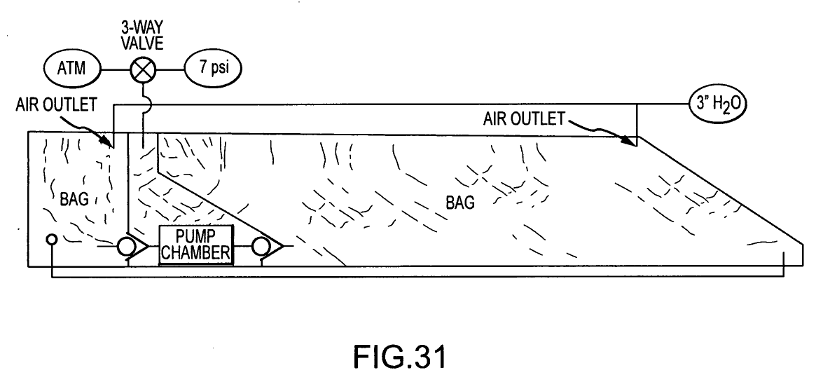

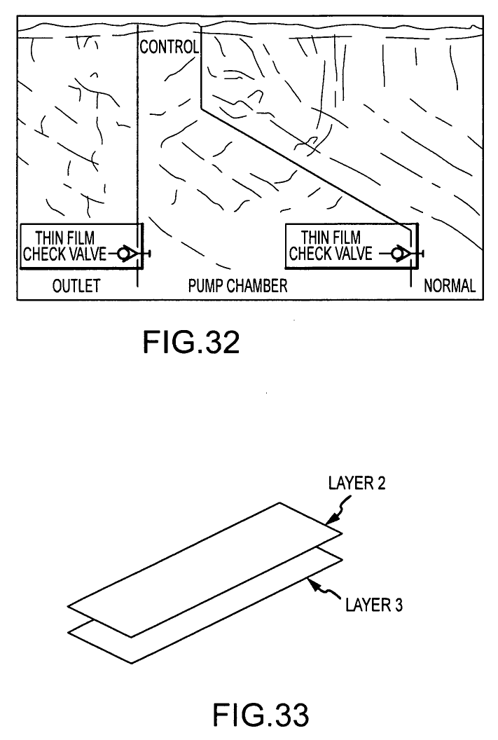

In certain embodiments of the present invention, the low shear air displacement pump is comprised largely of plastic film. Photobioreactor growth chambers may be formed of a flexible plastic film. FIG. 31 illustrates a growth chamber bag in which is incorporated a film-based air displacement pump, according to embodiments of the present invention. As shown in FIGS. 31 and 32, the pump chamber is incorporated directly into the plastic film that forms the photobioreactor growth chamber, by attachment of various elements and by sealing the edges of the pump chamber, for example by heat welding the plastic film. Check valves may be attached to the bottom of the film-based air displacement pump to regulate unidirectional water flow into and out of the pump chamber. A three-way valve or other regulatory mechanisms may be attached to the top of the pump chamber to regulate the flow of air into and out of the pump chamber.

According to some embodiments of the present invention, the operation of the film-based low shear air displacement pump is largely as described above with respect to FIGS. 1-4, 11, and 23. According to some embodiments of the present invention, a plastic loop, spring, spring-like element, or a like mechanical device is used to maintain an internal volume of the film-based pump chamber and/or bioreactor when surrounding hydrostatic pressure (e.g. when the film-based pump chamber is submerged in water) is greater than an internal pressure of the pump chamber. According to some embodiments of the present invention, the film bag structure itself is designed to assume an open volume shape. According to other embodiments of the present invention, the vent pressure of the pump chamber is higher than the hydrostatic pressure acting on the liquid within the pump chamber to maintain an internal volume in the pump chamber even during the fluid exhaust cycle.

Air in the pump chamber is vented to atmosphere or otherwise collected through the three-way valve. As air is released from the pump chamber, the inlet check valve opens and the pump chamber fills with the liquid growth medium in the photobioreactor chamber, upstream from the air displacement pump. Once the pump chamber has filled with liquid, the three-way valve is switched to allow pressurized air (at a pressure of seven pounds per square inch in some embodiments) to enter the pump chamber. This increase in pressure opens the outlet check valve and water is displaced by the air, out of the pump chamber and into the downstream side of the photobioreactor growth chamber.

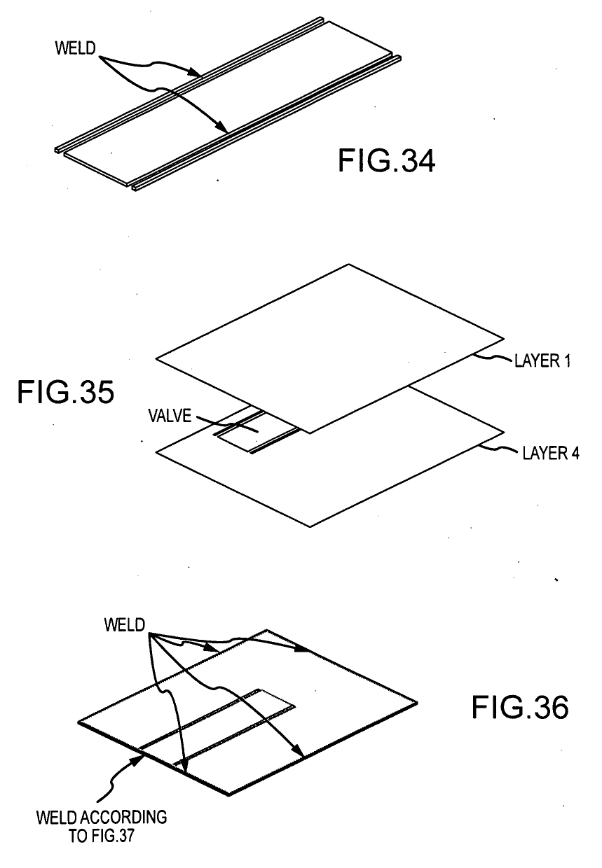

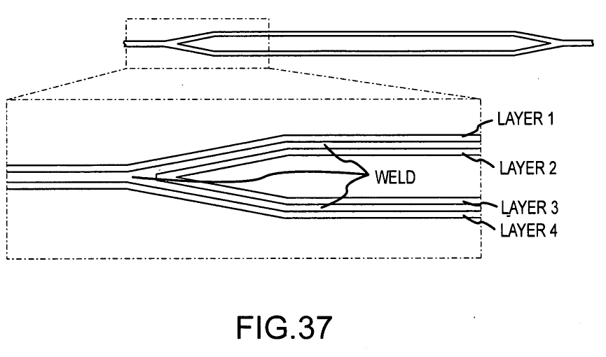

FIGS. 33-37 illustrate additional details regarding construction of a thin film check valve, which may be incorporated into the film-based air displacement pump. As shown in FIG. 33, the thin film check valve may include two layers. FIG. 34 illustrates that the layers may be welded along the long edges, and FIG. 35 illustrates that the valve may be placed into the center of the bag layers, according to embodiments of the present invention. FIG. 36 illustrates that the thin film check valve layers may be welded along their long edges, with the opening to the thin film check valve being welded as illustrated in FIG. 37, according to embodiments of the present invention.

According to such embodiments of the present invention, the thin film check valve assembly is comprised of four layers of plastic, two for the valve itself, and two for the pump chamber it is integral with. Pressure in the bag located between the outer bag layers keeps the thin film valve shut by putting external pressure against the inner valve layers. The valve opens and fluid enters the bag when the fluid is at a greater pressure than the pressure in the bag. According to some embodiments of the present invention, the distance between welds (forming the tubes) in the pump chamber is not more than three inches, in order to minimize stresses in the weld. Pressures less than seven pounds per square inch may be used as a pressurized gas source; according to some embodiments, a higher pressure gas source may be significantly throttled (e.g. throttled across a manual two-way valve) in order to keep the pump chamber from emptying too fast. And although FIG. 31 illustrates a ¾ inch flexible hose connecting the right and left ends of the bag, a larger diameter hose, such as, for example, a two inch diameter hose, may be used, according to embodiments of the present invention.

Any species of algae or photosynthetic microorganism may be grown in a photobioreactor and pumped using a low shear pump. For example, Tetraselmis suecica, UTEX 2286 and NREL/Hawaii TETRA 01, Tetraselmis chuii, Nannochloropsis oculata UTEX 2164, CCMP 525, Nannochloropsis sp. UTEX 2341, Nannochloropsis salina NANNO 01 NREL/Hawaii, CCMP 1776, 1777, 1776, Chlorella salina SAG 8.86, Chlorella protothecoides UTEX 25, Chlorella ellipsoidea UTEX 20 or several strains of Dunaliella tertiolecta (UTEX LB999, DCCBC5, ATCC 30929) and Dunaliella salina, Nannochloropsis oculata, Nannochloropsis gaditana, Nannochloropsis salina, Tetraselmis suecica, Tetraselmis chuii, Nannochloropsis sp., Chlorella salina, Chlorella protothecoides, Chlorella ellipsoidea, Dunaliella tertiolecta, Dunaliella salina, Phaeodactulum tricornutum, Botrycoccus braunii, Chlorella emersonii, Chlorella minutissima, Chlorella pyrenoidosa, Chlorella sorokiniana, Chlorella vulgaris, Chroomonas salina, Cyclotella cryptica, Cyclotella sp., Euglena gracilis, Gymnodinium nelsoni, Haematococcus pluvialis, Isochrysis galbana, Monoraphidium minutum, Monoraphidium sp., Neochloris oleoabundans, Nitzschia laevis, Onoraphidium sp., Pavlova lutheri, Phaeodactylum tricornutum, Porphyridium cruentum, Scenedesmus obliquuus, Scenedesmus quadricaula Scenedesmus sp., Stichococcus bacillaris, Spirulina platensis, Thalassiosira sp. Isochrysis sp., Phaeocystis, Nannochloris, Aureococcus, Prochlorococcus, Chlamydomonas, Synechococcus, Synechococcus, sp., Synechococcus elongates, Anacystis sp., Anacystis nidulans., Picochloroum oklahomensis, Stichococcus minor, Picocystis sp., Chlorella sp., Dunaliella sp., Dunaliella bardawil may be grown, either separately or as a mixture of species, in a bioreactor 102 and pumped using a low shear air displacement pump, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A pump system comprising:
a bioreactor comprising a media;
a gas vent line;
a pressurized gas line;
a valve system in fluid communication with the gas vent line and the pressurized gas line;
a fluid outlet;
a pump chamber in fluid communication with the bioreactor, the valve system, and the fluid outlet, wherein the valve system is configured to switch fluid communication with the pump chamber between the gas vent line and the pressurized gas line, and wherein a lowest level of the pump chamber is lower than a highest level of the bioreactor;
a first valve configured to permit one-way flow from the bioreactor to the pump chamber; and
a second valve configured to permit one-way flow from the pump chamber through the fluid outlet.

2. The pump system of claim 1, wherein one or more of the first and second valves is a check valve.

3. The pump system of claim 2, wherein one or more of the check valves is a thin film check valve.

4. The pump system of claim 3, wherein the bioreactor is a thin film photobioreactor.

5. The pump system of claim 1, wherein the thin film photobioreactor is a first thin film photobioreactor, the pump system further comprising a second thin film photobioreactor, wherein the second thin film photobioreactor is in fluid communication with the fluid outlet.

6. The pump system of claim 1, wherein one or more of the first and second valves is a solenoid valve.

7. The pump system of claim 1, wherein the bioreactor is a first bioreactor, the pump system further comprising a second bioreactor, wherein the second bioreactor is in fluid communication with the fluid outlet.

8. The pump system of claim 1, wherein the valve system is a dual-position, three-way solenoid valve.

9. The pump system of claim 1, wherein the valve system comprises a first two-position, two-way solenoid valve in the gas vent line and a second two-position, two-way solenoid valve in the pressurized gas line.

10. The pump system of claim 1, wherein a highest level of the pump chamber is higher than a media level of the bioreactor, to permit liquid level equilibrium between the pump chamber and the bioreactor when the pump chamber is in fluid communication with the gas vent line.

11. The pump system of claim 1, further comprising a regulated pressurized gas source in fluid communication with the pressurized gas line.

12. The pump system of claim 1, wherein the pump chamber is a first pump chamber, wherein the valve system is a first valve system, the pump system further comprising:
a second valve system in fluid communication with the gas vent line and the pressurized gas line; and
a second pump chamber in fluid communication with the bioreactor, the second valve system, and the fluid outlet, wherein the second valve system is configured to switch fluid communication with the second pump chamber between the gas vent line and the pressurized gas line, and wherein a lowest level of the second pump chamber is lower than a highest level of the bioreactor.

13. The pump system of claim 12, further comprising a controller operatively associated with the first and second valve systems, wherein the controller is configured to permit fluid communication with one of the first and second pump chambers to the gas vent line while permitting fluid communication with the other of the first and second pump chambers to the pressurized gas line.

14. The pump system of claim 12, further comprising:
a third valve configured to permit one-way flow from the bioreactor to the second pump chamber; and
a fourth valve configured to permit one-way flow from the second pump chamber through the fluid outlet.

15. The pump system of claim 1, wherein the pump chamber is a first pump chamber, wherein the valve system is a first valve system, wherein the gas vent line is a first gas vent line, wherein the pressurized gas line is a first pressurized gas line, and wherein the fluid outlet is a first fluid outlet, the pump system further comprising:
a second gas vent line;
a second pressurized gas line;
a second valve system in fluid communication with the second gas vent line and the second pressurized gas line;
a second fluid outlet; and
a second pump chamber in fluid communication with the bioreactor, the second valve system, and the second fluid outlet, wherein the second valve system is configured to switch fluid communication with the second pump chamber between the second gas vent line and the second pressurized gas line, and wherein a lowest level of the second pump chamber is lower than a highest level of the bioreactor.

16. The pump system of claim 15, wherein the second gas vent line is in fluid communication with the first gas vent line, wherein the second pressurized gas line is in fluid communication with the first pressurized gas line, and wherein the second fluid outlet is in fluid communication with the first fluid outlet.

17. The pump system of claim 13, wherein the first pump chamber comprises a liquid level sensor configured to sense a liquid level in the first pump chamber, and wherein the controller is operatively associated with the liquid level sensor and is further configured to switch fluid communication with the first pump chamber from the gas vent line to the pressurized gas line when the liquid level reaches a predetermined liquid level.

18. A method for pumping bioreactor media at low shear, the method comprising:
connecting a pump chamber with a bioreactor containing media;
placing a valve inline between the pump chamber and the bioreactor, the valve configured to permit one-way flow from the bioreactor to the pump chamber;
arranging the bioreactor and the pump chamber such that a media level in the bioreactor is higher than a liquid level in the pump chamber; and
venting the pump chamber to begin flow of the media from the bioreactor, through the valve, and into the pump chamber, wherein the flow of the media is at least partially caused by a difference in head pressure between the bioreactor and the pump chamber.

19. The method of claim 18, further comprising:
closing a vent of the pump chamber; and
applying a pressurized gas source to the pump chamber to begin flow of the media out of the pump chamber.

20. The method of claim 19, wherein the pump chamber is a first pump chamber, wherein the valve is a first valve, wherein the liquid level is a first liquid level, the method further comprising:
connecting a second pump chamber with the bioreactor;
placing a second valve inline between the second pump chamber and the bioreactor, the second valve configured to permit one-way flow from the bioreactor to the second pump chamber;
arranging the bioreactor and the second pump chamber such that the media level in the bioreactor is higher than a second liquid level in the second pump chamber;
alternately venting the first pump chamber when the pressurized gas source is applied to the second pump chamber and venting the second pump chamber when the pressurized gas source is applied to the first pump chamber, to permit a substantially continuous flow of media out of the bioreactor.

21. A pump system comprising:
a bioreactor comprising a media;
a gas vent line;
a pressurized gas line;
a valve system in fluid communication with one of the gas vent line and the pressurized gas line;
a fluid outlet;
a pump chamber in fluid communication with the bioreactor, the valve system, and the fluid outlet, wherein the valve system is configured to open or close fluid communication between the pump chamber and the one of the gas vent line and the pressurized gas line; and
a valve configured to permit one-way flow from the bioreactor to the pump chamber or from the pump chamber to the fluid outlet.

22. The pump system of claim 21, wherein the valve system is in fluid communication with the gas vent line, and the pressurized gas line is in fluid communication with the pump chamber.

23. The pump system of claim 21, wherein the valve system is in fluid communication with the pressurized gas line, and the gas vent line is in fluid communication with the pump chamber.

24. The pump system of claim 21, wherein the pump chamber is a first pump chamber, wherein the valve system is a first valve system, wherein the one of the gas vent line and the pressurized gas line is a first one of the gas vent line and the pressurized gas line, the pump system further comprising:
a second valve system in fluid communication with a second one of the gas vent line and the pressurized gas line; and
a second pump chamber in fluid communication with the bioreactor, the second valve system, and the fluid outlet, wherein the second valve system is configured to open or close fluid communication between the second pump chamber and the second one of the gas vent line and the pressurized gas line.

25. The pump system of claim 21, wherein the pump chamber is a first pump chamber, wherein the valve system is a first valve system, wherein the one of the gas vent line and the pressurized gas line is a first one of the gas vent line and the pressurized gas line, wherein the gas vent line is a first gas vent line, wherein the pressurized gas line is a first pressurized gas line, and wherein the fluid outlet is a first fluid outlet, the pump system further comprising:
a second gas vent line;
a second pressurized gas line;
a second valve system in fluid communication with a second one of the second gas vent line and the second pressurized gas line;
a second fluid outlet; and
a second pump chamber in fluid communication with the bioreactor, the second valve system, and the second fluid outlet, wherein the second valve system is configured to open or close fluid communication between the second pump chamber and the second one of the second gas vent line and the second pressurized gas line.
